(19) United States
(12) Patent Application Publication
(10) Pub. No.: US 2021/0196186 A1
(43) Pub. Date: Jul. 1, 2021

(54) ACNE DETECTION USING IMAGE ANALYSIS

(71) Applicant: L'OREAL, Paris (FR)

(72) Inventors: Kyle Yeates, Redmond, WA (US); Ozgur Yildirim, Redmond, WA (US)

(73) Assignee: L'OREAL, Paris (FR)

(21) Appl. No.: 17/138,393

(22) Filed: Dec. 30, 2020

Related U.S. Application Data
(60) Provisional application No. 62/955,128, filed on Dec. 30, 2019.

(51) Int. Cl. A61B 5/00; A61B 5/103; G06T 7/00

(52) U.S. Cl. CPC A61B 5/445; G06T 2207/10024; A61B 5/7282; A61B 5/7485; A61B 5/486; A61B 5/1032; A61B 5/4842; A61B 5/7246; G06T 7/0016; A61B 5/7435; A61B 5/6898; A61B 5/7267; G06T 2207/30088; G06T 2207/30096; G06T 2200/24; A61B 5/0077

(57) ABSTRACT

Examples of methodologies and technologies for determining changes in one or more skin conditions of a user over time are described herein. Any changes in skin conditions over time may be used as a diagnosis and/or treatment aid for a physician. Any changes in skin conditions over time may be also used in a computer implemented method that provides diagnosis and/or treatment recommendations.

### CROSS-REFERENCE TO RELATED APPLICATIONS

This application claims the benefit of U.S. Provisional Application No. 62/955,128, filed Dec. 30, 2019, the disclosure of which is incorporated herein in its entirety.

### TECHNICAL FIELD

Embodiments of the present disclosure relate to image processing. In some embodiments, such image processing techniques are employed for skin condition detection and/or treatment.

### SUMMARY OF DISCLOSURE

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with an aspect of the disclosure, examples of a computer implemented method for determining changes in a skin condition of a subject is provided. In an embodiment, the computer implement method comprises obtaining a plurality of images of an area of interest associated with the subject, the plurality of images taken sequentially over time, wherein each image taken is separated in time by a time period; and determining one or more differences between the plurality of images.

In any embodiment, the computer implemented method may further comprise generating an image map of the area of interest, the image map indicative of the differences between the plurality of images.

In any embodiment, the computer implemented method may further comprise determining a skin condition based on the image map.

In any embodiment, the image map indicates changes in one or more of a size, a shape, a color, and uniformity of an object contained in the area of interest.

In any embodiment, the computer implemented method may further comprise recommending one of a treatment or a product based on the determined skin condition.

In any embodiment, the skin condition is selected from a group consisting of dermatitis, eczema, acne, and psoriasis.

In any embodiment, the time period is selected from the group consisting of 24 hours, one week, one month, two months, three months, four months, five months, and six months.

In any embodiment, the computer implemented method may further comprise notifying the user that a change has been detected if the difference detected is greater than a preselected threshold value.

In any embodiment, the computer implemented method may further comprise determining the area of interest based at least one the captured images.

In accordance with another aspect of the disclosure, examples of a system for determining changes in a skin condition of a subject is provided. In one embodiment the system comprises a camera configured to capture one or more images; and one or more processing engines including circuitry configured to: cause the camera to capture one or more images of an area of interest associated with the subject, the one or more images taken sequentially over time so as to obtain a plurality of images separated in time by a time period selected from the group consisting of 24 hours, one week, one month, two months, three months, four months, five months, and six months, and one year; determine one or more differences between the captured images, the differences indicative of changes in one or more of a size, a shape, a color, and uniformity of an object contained in the area of interest; and determine a skin condition based on the determined differences or flagging the object for subsequent analysis if the differences are greater than a preselected threshold.

In any embodiment of the system, the one or more processing engines include circuitry configured to: determine the skin condition based on the determined differences; and recommend a treatment protocol or a product based on the determined skin condition.

In any embodiment of the system, the one or more processing engines includes circuitry configured to determine changes in one or more of: size, shape, color, uniformity of an existing lesion, detect new lesions, detect the absence of previously detected lesion(s), or detect a progression of a lesion.

In any embodiment of the system, the one or more processing engines includes circuitry configured to: detect a progression of a lesion from the detected differences in the plurality of images; and determine one or more stages of the lesion based on the detected progression of the lesion.

In any embodiment of the system, the one or more processing engines includes: a user interface engine including circuitry configured to cause the camera to capture the plurality of images; an image analysis engine including circuitry for comparing two or more images using a similar/difference algorithm to determine one or more differences between the images; and a skin condition engine including circuity configured for analyzing an image map of the determined one or more differences to locate a lesion, and for determining the stage of the lesion located in the image map.

In any embodiment of the system, the one or more processing engines further includes: a recommendation engine including circuity configured to recommend a treatment protocol and/or product for each region based at least on the determined skin condition.

In any embodiment of the system, the skin condition is selected from a group consisting of dermatitis, eczema, acne, and psoriasis.

In accordance with another aspect of the disclosure, examples of a computer-implemented method are provided for determining changes in a skin condition of a subject. In an embodiment, the method comprises obtaining a plurality of images of an area of interest associated with the subject, the plurality of images taken sequentially over a time with each taken image separated in time by a time period; determining a skin condition based on least the plurality of images; determining at least one product recommendation based on at least the determined skin condition; and providing the at least one product recommendation to the subject.

In any embodiment of the computer implemented method, obtaining, by a first computing device, a plurality of images of an area of interest associated with the subject includes capturing, by a camera of a first computing device, the plurality of images.

In any embodiment of the computer implemented method, determining a skin condition based on least the plurality of images or the determining at least one product recommendation based on at least the determined skin condition is carried out by a second computing device remote from the first computing device.

In any embodiment of the computer implemented method, the skin condition is selected from a group consisting of dermatitis, eczema, acne, and psoriasis.

### DESCRIPTION OF THE DRAWINGS

The foregoing aspects and many of the attendant advantages of disclosed subject matter will become more readily appreciated as the same become better understood by reference to the following detailed description, when taken in conjunction with the accompanying drawings, wherein:

FIG. 1 is a schematic diagram that illustrates a non-limiting example of a system for detecting and/or diagnosing skin conditions of a user according to an aspect of the present disclosure;

FIG. 2 is a block diagram that illustrates a non-limiting example of a mobile computing device according to an aspect of the present disclosure;

FIG. 3 is a block diagram that illustrates a non-limiting example of a server computing device according to an aspect of the present disclosure;

FIG. 4 is a block diagram that illustrates a non-limiting example of a computing device appropriate for use as a computing device with embodiments of the present disclosure.

FIG. 5 is a flowchart that illustrates a non-limiting example of a method for detecting and/or diagnosing a skin condition according to an aspect of the present disclosure.

### DETAILED DESCRIPTION

Examples of methodologies and technologies for determining changes in one or more skin conditions of a user over time are described herein. Any changes in skin conditions over time may be used as an diagnosis and/or treatment aid for a physician. Any changes in skin conditions over time may be also used in a computer implemented method that provides diagnosis and/or treatment recommendations.

Thus, in the following description, numerous specific details are set forth to provide a thorough understanding of the examples. One skilled in the relevant art will recognize; however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one example” or “one embodiment” means that a particular feature, structure, or characteristic described in connection with the example is included in at least one example of the present invention. Thus, the appearances of the phrases “in one example” or “in one embodiment” in various places throughout this specification are not necessarily all referring to the same example. Furthermore, the particular features, structures, or characteristics may be combined in any suitable manner in one or more examples.

The disclosed subject matter provides examples of systems and methods for detecting a skin condition, such as acne, by looking at multiple images of a user taken at different points in time (e.g., once a day for 1-2 weeks, once a day for a month, etc.) and using image processing techniques to detect changes of size, shape, color, uniformity, etc., of areas of the image to determine whether the changes represent characteristics (e.g., blemishes) caused by a skin condition (e.g., acne). For example, the images can be captured by a camera of the consumer product (e.g., mobile phone, tablet, etc.) and then transferred to a computer system that stores the images for subsequent access and analysis. In some examples, the computer system is part of the consumer product (e.g., mobile phone, tablet, etc.). After a number of images are collected, the computer system compares the images for detecting changes in the images over time (e.g., from the earliest image to the latest image). If any changes are detected, skin condition analysis can be carried out in some embodiments to determine how many acne blemishes exist, how severe the user's acne is, what stage of acne each blemish is in, etc.

With this information, the system and methods in some examples can recommend a treatment based on results of the skin condition analysis. The treatment recommendation can include one or more treatment protocols and may include, for example, one or more product recommendations. In some examples, the systems and methods can track the efficacy of the recommendation and can train the system for improved recommendations in subsequent uses.

In general, features on the face, for example, are static (e.g., location of nose, lips, chin, moles, freckles, etc.) relative to acne blemishes. Acne blemishes last anywhere from 5-10 days to months, and during this span the acne blemish follows an understood trajectory (e.g., blocked pore, black head, white head, papule, pustule, lesion, scar). Each stage of the blemish has unique colors and sizes relative to the other stages. By understanding the overall lifespan of the acne blemish and taking multiple, sequential images of the face (e.g., once a day, once a week, etc.), a skin condition (e.g., acne, etc.) map or profile can be generated.

For example, multiple images of an area of interest of the user taken over time can be analyzed via image processing techniques for determining changes in skin condition(s). If the changes to certain areas (e.g., pixel groups) of the images match, for example, the progression of a known skin condition (e.g., an acne blemish), the systems and methods in some examples identify groups of pixels as a blemish and can create an acne profile of the user associated with this area of interest. The profile may include, for example, assignment of an acne stage(s) to each blemish or sections thereof. This profile can then be matched to suggested products and treatment protocols to address the skin condition. While the face is described in some embodiments, other body locations of the user can be monitored, such as the back, the chest, arms, etc. Of course, multiple areas of interest can be analyzed, and an acne profile can be generated for each area of interest.

In other examples, the system and methods again capture images of an area of interest (e.g., the back) taken at different points in time. In these examples, the time period is extended (e.g., every 6 months, every year). The images are then transferred to a computer system that stores the images for subsequent access and analysis. In some examples, the computer system is part of the image capture device (e.g., mobile phone, tablet, etc.).

After a number of images are collected over time, the computer system can compare the images to identify, for example, new lesions (e.g. moles, sun spots, aging spots, etc.) that did not exist before, or flag lesions that underwent a change (e.g., size, shape, color, uniformity etc.) greater than a predetermined threshold (e.g., 2-5% change). With the computer system, suspicious lesions can be identified and flagged for closer examination by a dermatologist, or other methods. With the lesions identified by the system, the dermatologist will be more able to identify and focus on the most concerning lesions.

Accordingly, examples of the systems and methods provide an extremely powerful tool that can be deployed on a simple consumer product, such as a smart phone, tablet, etc., with optional cloud or server storage systems for assisting dermatologists in identifying potential problems, such as cancer. And since the systems and methods can be deployed in consumer products owed or accessible to most users, these systems and methods can to utilized to assist the user in tracking the changes over time (e.g., reduction) of individual lesions (blemishes, acne lesions, dark spots, etc.) to demonstrate the effectiveness of their cosmetic interventions and to provide encouragement to continue such treatment by demonstrating the actual changes over time. If such treatment is shown by the systems and methods of the present disclosure to be ineffective, the user is able to change treatment protocols sooner than without such tools.

In some examples, the methodologies and technologies are carried out by a computing system that includes, for example, a handheld smart device (e.g., a smart phone, tablet, laptop, game console, etc.) with a camera and memory. An optional cloud data store can be accessed by the system for storage of images of the user at different time points with appropriate metadata (e.g., date, user ID, user annotations etc.). The computing system also includes an image processing algorithm or engine that is either local to the handheld smart device or remote to the handheld smart device (e.g., server/cloud system) for analyzing the captured images.

In some embodiments, the image processing algorithm or engine compares and interprets the gross changes of lesions over time to determine and flag (e.g., identify, highlight, mark, etc.) a subset of lesions that are categorized as “suspicious.” The system may also notify the subject of when such lesions are flagged. Such flagged lesions can be further analyzed by advanced algorithms or reviewed by a physician. In other embodiments, the image processing algorithm or engine compares and interprets the changes of lesions over time for generating an skin condition profile (e.g., acne profile). A user interface can be presented by the handheld smart device to aid the user in image capture, image storage, access to previously stored images, interaction with the analysis engines and to notify and/or display any lesions flagged as suspicious by the system.

In some examples, some methodologies and technologies of the disclosure are provided to a user as a computer application (i.e., an “App”) through a mobile computing device, such as a smart phone, a tablet, a wearable computing device, or other computing devices that are mobile and are configured to provide an App to a user. In other examples, the methodologies and technologies of the disclosure may be provided to a user on a computer device by way of a network, through the Internet, or directly through hardware configured to provide the methodologies and technologies to a user.

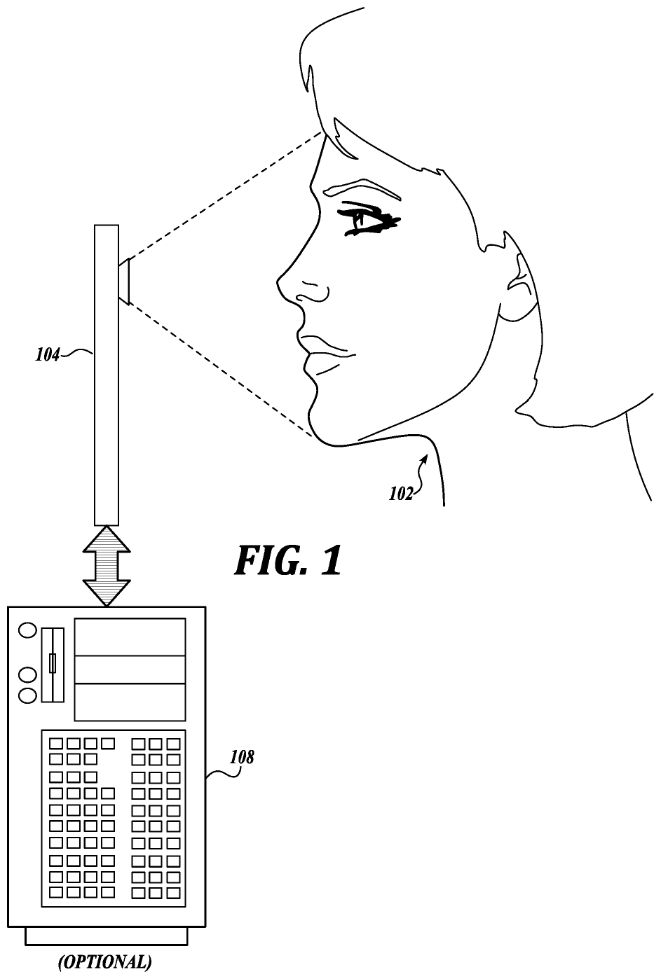

FIG. 1 is a schematic diagram that illustrates a non-limiting embodiment of a system for detecting changes in the skin condition of a user according to an aspect of the present disclosure. In the system 100, a user 102 interacts with a mobile computing device 104. The mobile computing device 104 may be used to capture one or more images of the user 102, from which at least one skin condition, such as acne, eczema, psoriasis, or suspicious lesion can be diagnosed. As will be described in more detail below, the mobile computing device 104 can be used to capture one or more image(s) of the user's area of interest (e.g., back, face, neck, etc.) at different points in time (e.g., once a week, once a month, once every six months, once a year, etc.)

In some embodiments, the mobile computing device 104 is used to process the collected images in order to determine changes of the area of interest over a selected period of time. The selected period of time can be, for example, one week, one month, one year, etc. In some embodiments, the results of the processed images can then be used for diagnostic purposes by a physician. For example, the results of the processed images may indicate a suspicious lesion. The physician can then use the results to determine whether a biopsy or other further analysis should be made.

In some other embodiments, the mobile computing device 104 analyzes the changes reflected in the processed images for determining skin conditions associated with the area of interest. With this skin condition information, the mobile computing device may also be used for determining a product recommendation, treatment protocol, etc., to be presented to the user 102. The efficacy of the treatment protocol, product usage, etc., may then be tracked with subsequent image capture and analysis by the mobile computing device 104.

As will be described in more detail below, some of the functionality of the mobile computing device 104 can be additionally or alternatively carried out at an optional server computing device 108. For example, the mobile computing device 104 in some embodiments transmits the captured images to the server computing device 108 via a network 110 for image processing and/or storage. In some embodiments, the network 110 may include any suitable wireless communication technology (including but not limited to Wi-Fi, WiMAX, Bluetooth, 2G, 3G, 4G, 5G, and LTE), wired communication technology (including but not limited to Ethernet, USB, and FireWire), or combinations thereof.

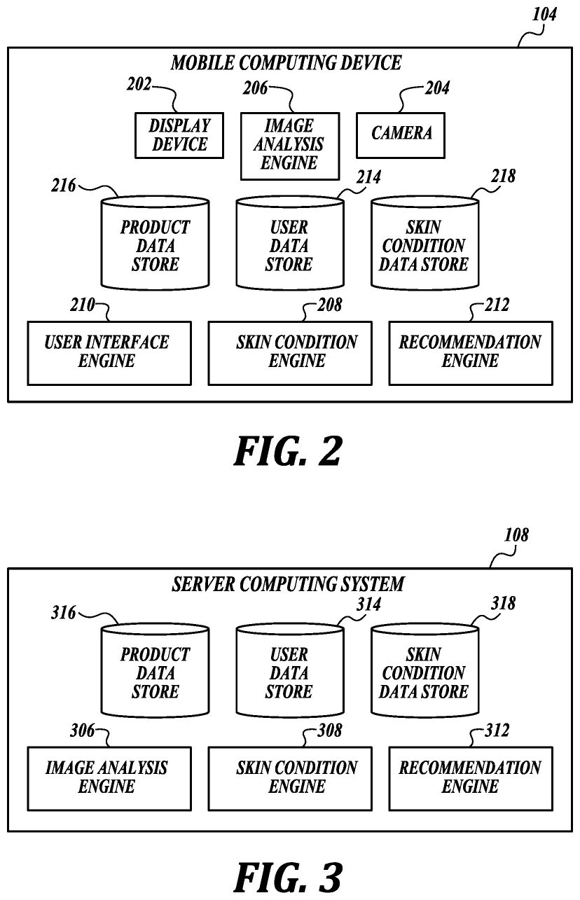

FIG. 2 is a block diagram that illustrates a non-limiting example embodiment of a system that includes a mobile computing device 104 according to an aspect of the present disclosure. The mobile computing device 104 is configured to collect information from a user 102 in the form of images of an area of interest. The area of interest can be a specific body part of the user, such as the back, face, arm, neck, etc., or can be region(s) thereof, such as the forehead, chin, or nose of the face, the shoulder, dorsum, or lumbus of the back, etc.

In some embodiments, the mobile computing device 104 may be a smartphone. In some embodiments, the mobile computing device 104 may be any other type of computing device having the illustrated components, including but not limited to a tablet computing device or a laptop computing device. In some embodiments, the mobile computing device 104 may not be mobile, but may instead by a stationary computing device such as a desktop computing device or computer kiosk. In some embodiments, the illustrated components of the mobile computing device 104 may be within a single housing. In some embodiments, the illustrated components of the mobile computing device 104 may be in separate housings that are communicatively coupled through wired or wireless connections (such as a laptop computing device with an external camera connected via a USB cable). The mobile computing device 104 also includes other components that are not illustrated, including but not limited to one or more processors, a non-transitory computer-readable medium, a power source, and one or more communication interfaces.

As shown, the mobile computing device 104 includes a display device 202, a camera 204, an image analysis engine 206, a skin condition engine 208, a user interface engine 210, a recommendation engine 212, and one or more data stores, such as a user data store 214, a product data store 216 and/or skin condition data store 218. Each of these components will be described in turn.

In some embodiments, the display device 202 is an LED display, an OLED display, or another type of display for presenting a user interface. In some embodiments, the display device 202 may be combined with or include a touch-sensitive layer, such that a user 102 may interact with a user interface presented on the display device 202 by touching the display. In some embodiments, a separate user interface device, including but not limited to a mouse, a keyboard, or a stylus, may be used to interact with a user interface presented on the display device 202.

In some embodiments, the user interface engine 210 is configured to present a user interface on the display device 202. In some embodiments, the user interface engine 210 may be configured to use the camera 204 to capture images of the user 102. Of course, a separate image capture engine may also be employed to carry out at least some of the functionality of the user interface 210. The user interface presented on the display device 202 can aid the user in capturing images, storing the captured images, accessing the previously stored images, interacting with the other engines, etc. The user interface presented on the display device 202 can also present one or more lesions that were flagged as suspicious by the system, and can present a treatment protocol to the user 102 with or without product recommendations.

In some embodiments, the user interface engine 210 may also be configured to create a user profile. Information in the user profile may be stored in a data store, such as the user data store 214. Data generated and/or gathered by the system 100 (e.g., images, analysis data, statistical data, user activity data, or other data) may also be stored in the user data store 214 from each session when the user 102 utilizes the system 100. The user profile information may therefore incorporate information the user provides to the system through an input means, for example, such as a keyboard, a touchscreen, or any other input means. The user profile may farther incorporate information generated or gathered by the system 100, such as statistical results, recommendations, and may include information gathered from social network sites, such as Facebook™, Instagram, etc. The user may input information such as the user's name, the user's email address, social network information pertaining to the user, the user's age, user's area of interest, and any medications, topical creams or ointments, cosmetic products, treatment protocol, etc., currently used by the user, previously recommended treatments and/or products, etc.

In some embodiments, the camera 204 is any suitable type of digital camera that is used by the mobile computing device 104. In some embodiments, the mobile computing device 104 may include more than one camera 204, such as a front-facing camera and a rear-facing camera. Generally herein, any reference to images being utilized by embodiments of the present disclosure should be understood to reference video, images (one or more images), or video and images (one or more images), as the present disclosure is operable to utilize video, images (one or more images), or video and images (one or more images) in its methods and systems described herein.

In some embodiments, the mobile computing device 104 may use an image capture engine (not shown) to capture images of the user. In some embodiments, the image capture engine is part of the user interface engine 210. In an embodiment, the image capture engine is configured to capture one or more images of an area of interest. The area of interest can be for example the back, the face, the neck, the chest, or sections thereof, of the user 102. The images can be captured by the user 102 as a “selfie,” or the mobile computing device 104 can be used by a third party for capturing images of a user 102. In some embodiments, the image capture engine timestamps the captured image(s) and stores the images according to the user profile with other data, such as flash/camera settings. The image capture engine may also send the images with the associated information to the server computer device 108 for storage, optional processing, and subsequent retrieval, as will be described in more detail below.

In some embodiments, the image analysis engine 206 is configured to compare two or more images. The image analysis engine 206 checks the timestamps of the images and runs a similar/difference algorithm or image processing routine. In some embodiments, the similar/difference algorithm determines or detects changes in size, shape, color, uniformity, etc., of existing lesions (e.g., moles, acne, dark sports, etc.), detects new lesions, detects the absence of previously detected lesions, detects a progression of a lesion, etc. In some embodiments, image analysis engine 206 compares and interprets the gross changes of the lesions over time so as to decide and flag (e.g., identify, highlight, mark, etc.) a subset of lesions as “suspicious.” The lesions that are flagged as suspicious have changed in size, shape, color, uniformity, etc., an amount greater than a predetermined threshold. This subset of lesions can be highlighted on the image, represented in a skin condition map or profile, etc. In some embodiments, the image analysis engine 206 can identify the changes in the images as acne blemishes, which can also be highlighted on the image, represented in a skin condition map or profile, etc.

In some embodiments, the skin condition engine 208 is configured to analyze, for example, the skin condition map or profile, and can determine, for example, the stages of acne for each region of the image. In doing so, the skin condition engine 208 can access data from the skin condition data store 218. In some embodiments, the skin condition engine 208 identifies a progression of a skin condition, such as acne (e.g., determined from an analyses of the images). If the changes to certain areas (e.g., pixel groups) of the images match, for example, the progression of a known skin condition (e.g., an acne blemish) accessed from the skin condition data store 218, the skin condition engine 208 can identify these groups of pixels as a blemish and can assigned the blemish a skin condition level (e.g., acne stage, etc.). Of course, some of the functionality of the skin condition engine 208 can be shared or carried out by the image processing engine 206, and vice versa.

With the results of the analysis, the recommendation engine 212 in some embodiments is configured to recommend a treatment protocol and/or product (e.g., topical formula, such as an ointment, cream, lotion, etc.) for each region based at least on the determined skin condition (e. g., stage of acne, etc.). In doing so, the recommendation engine 212 can access data from the product data store 216 and/or the user data store 214. Any recommendation generated by the recommendation engine 212 can be presented to the user in any fashion via the user interface engine 210 on display 202.

Further details about the actions performed by each of these components are provided below.

“Engine” refers to refers to logic embodied in hardware or software instructions, which can be written in a programming language, such as C, C++, COBOL, JAVA™ PHP, Perl, HTML, CSS, JavaScript, VBScript, ASP, Microsoft .NET™, Go, and/or the like. An engine may be compiled into executable programs or written in interpreted programming languages. Software engines may be callable from other engines or from themselves. Generally, the engines described herein refer to logical modules that can be merged with other engines or can be divided into sub-engines. The engines can be stored in any type of computer-readable medium or computer storage device and be stored on and executed by one or more general purpose computers, thus creating a special purpose computer configured to provide the engine or the functionality thereof.

“Data store” refers to any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores, without departing from the scope of the present disclosure.

FIG. 3 is a block diagram that illustrates various components of a non-limiting example of an optional server computing device 108 according to an aspect of the present disclosure. In some embodiments, the server computing device 108 includes one or more computing devices that each include one or more processors, non-transitory computer-readable media, and network communication interfaces that are collectively configured to provide the components illustrated below. In some embodiments, the one or more computing devices that make up the server computing device 108 may be rack-mount computing devices, desktop computing devices, or computing devices of a cloud computing service.

In some embodiments, image processing and/or storage of the captured images can be additionally or alternatively carried out at an optional server computing device 108. In that regard, the server computing device 108 can receive captured and/or processed images from the mobile computing device 104 over the network 110 for processing and/or storage. As shown, the server computing device 108 optionally includes an image analysis engine 306, a skin condition engine 308, a recommendation engine 312, and one or more data stores, such as a user data store 314, a product data store 316 and/or skin condition data store 318. It will be appreciated that the image analysis engine 306, a skin condition engine 308, a recommendation engine 312, and one or more data stores, such as a user data store 314, a product data store 316 and/or skin condition data store 318 are substantially identical in structure and functionality as the image analysis engine 206, a skin condition engine 208, a recommendation engine 212, and one or more data stores, such as a user data store 214, a product data store 216 and/or skin condition data store 218 of the mobile computing device 104 illustrated in FIG. 2.

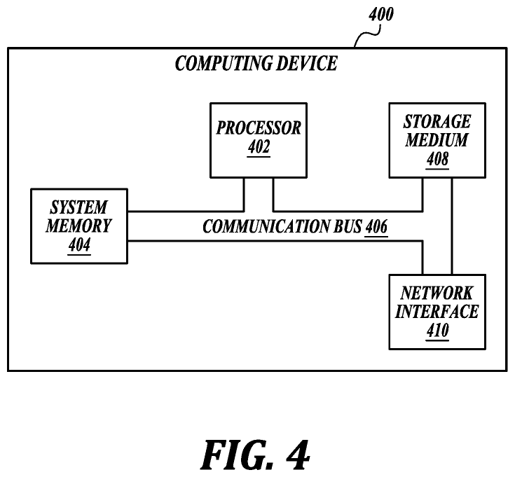

FIG. 4 is a block diagram that illustrates aspects of an exemplary computing device 400 appropriate for use as a computing device of the present disclosure. While multiple different types of computing devices were discussed above, the exemplary computing device 400 describes various elements that are common to many different types of computing devices. While FIG. 4 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Moreover, those of ordinary skill in the art and others will recognize that the computing device 400 may be any one of any number of currently available or yet to be developed devices.

In its most basic configuration, the computing device 400 includes at least one processor 402 and a system memory 404 connected by a communication bus 406. Depending on the exact configuration and type of device, the system memory 404 may be volatile or nonvolatile memory, such as read only memory (“ROM”), random access memory (“RAM”), EEPROM, flash memory, or similar memory technology. Those of ordinary skill in the art and others will recognize that system memory 404 typically stores data and/or program modules that are immediately accessible to and/or currently being operated on by the processor 402. In this regard, the processor 402 may serve as a computational center of the computing device 400 by supporting the execution of instructions.

As further illustrated in FIG. 4, the computing device 400 may include a network interface 410 comprising one or more components for communicating with other devices over a network. Embodiments of the present disclosure may access basic services that utilize the network interface 410 to perform communications using common network protocols. The network interface 410 may also include a wireless network interface configured to communicate via one or more wireless communication protocols, such as WIFI, 2G, 3G, LTE, WiMAX, Bluetooth, Bluetooth low energy, and/or the like. As will be appreciated by one of ordinary skill in the art, the network interface 410 illustrated in FIG. 4 may represent one or more wireless interfaces or physical communication interfaces described and illustrated above with respect to particular components of the computing device 400.

In the exemplary embodiment depicted in FIG. 4, the computing device 400 also includes a storage medium 408. However, services may be accessed using a computing device that does not include means for persisting data to a local storage medium. Therefore, the storage medium 408 depicted in FIG. 4 is represented with a dashed line to indicate that the storage medium 408 is optional. In any event, the storage medium 408 may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

As used herein, the term “computer-readable medium” includes volatile and non-volatile and removable and non-removable media implemented in any method or technology capable of storing information, such as computer readable instructions, data structures, program modules, or other data. In this regard, the system memory 404 and storage medium 408 depicted in FIG. 4 are merely examples of computer-readable media.

Suitable implementations of computing devices that include a processor 402, system memory 404, communication bus 406, storage medium 408, and network interface 410 are known and commercially available. For ease of illustration and because it is not important for an understanding of the claimed subject matter, FIG. 4 does not show some of the typical components of many computing devices. In this regard, the computing device 400 may include input devices, such as a keyboard, keypad, mouse, microphone, touch input device, touch screen, tablet, and/or the like. Such input devices may be coupled to the computing device 400 by wired or wireless connections including RF, infrared, serial, parallel, Bluetooth, Bluetooth low energy, USB, or other suitable connections protocols using wireless or physical connections. Similarly, the computing device 400 may also include output devices such as a display, speakers, printer, etc. Since these devices are well known in the art, they are not illustrated or described further herein.

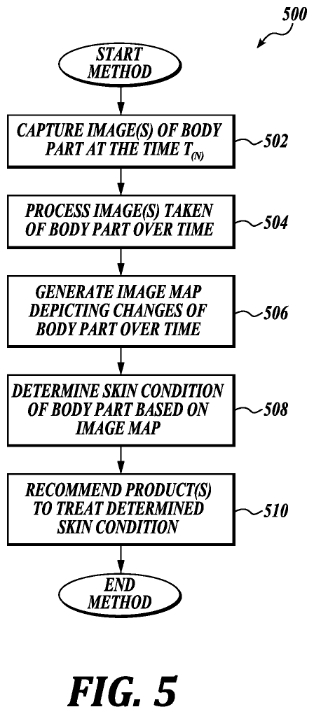

FIG. 5 is a flowchart that illustrates a non-limiting example embodiment of a method 500 for determining changes in skin conditions of a user according to various aspects of the present disclosure. In some embodiments, the method 500 also analyzes the changes in skin conditions and optionally recommends a treatment protocol and/or product to treat the user 102. It will be appreciated that the following method steps can be. carried out in any order or at the same time, unless an order is set forth in an express manner or understood in view of the context of the various operation(s). Additional process steps can also be carried out. Of course, some of the method steps can be combined or omitted in example embodiments.

From a start block, the method 500 proceeds to block 502, where a mobile computing device 104 captures image(s) of the user 102 at a time (T₁, T₂, Tₙ). In some embodiments, the mobile computing device 104 uses the camera 204 to capture at least one image. In some embodiments, more than one image with different lighting conditions may be captured in order to allow an accurate color determination to be generated. In some embodiments, the captured image is of an area of interest to the user 102. For example, the area of interest can be one of face, the neck, the back, etc., for tracking lesions, such as moles, sun spots, acne, eczema, etc., skin condition analysis, etc.

The one or more images can be stored in the user data store 214 at the mobile computing device 104 and/or server computer 108. When stored, additional data collected at the time of image capture can be associated with the images. For example, each image is time stamped, and may include other information, such as camera settings, flash settings, etc., area of interest captured, etc.

For new users, the user interface engine 210 can be used to create a user profile, as described above. At the time of image capture, the user interface engine 210 may query the user to enter the intended location (e.g., back, face, arm, neck, etc.) so that the captured image can be associated with the user's area of interest. The area of interest can be a specific body part of the user, such as the back, face, arm, neck, etc., or can be regions thereof, such as the forehead, chin, or nose of the face, the shoulder, dorsum, or lumbus of the back, etc. If the user has more than one area of interest, the user interface engine 210 can be repeatedly used until all images are captured. The captured images are stored in the user data store 214. If stored at the server computer 108 in user data store 314, the mobile computing device 104 can transmit the images over the network 110.

Images of the same area of interest are then captured sequentially over a period of time (T₁, T₂, T₃, Tₙ) at block 502. For example, the images can be captured daily, weekly, bi-weekly, monthly, bi-monthly, semi-annually, annually, etc.

Of course, the period of image capture can change during observation of the area of interest. For example, if an area of interest is flagged by the system, the user is notified by the system or if the user notices changes when reviewing one or more of the captured images, the frequency of image capture can be adjusted accordingly.

Next, at block 504, the images captured over a period of time are processed by the image analysis engine 206 of the mobile computing device 104 or the image analysis engine 306 of the server computing device 108. In that regard, the images collected over time are processed, for example, to detect differences or changes in the images by comparing each image to the other images. In some embodiments, the image analysis engine is initiated by user input (e.g., via user interface 210). In other embodiments, the image analysis engine may automatically analyze the images once the images are stored in user data store 214 and/or 314. If differences are determined, the image analysis engine is configured to notify the user. For example, if the determined differences are greater than a preset threshold value, the user is notified. Notification can be carried out via email, text message, banner notification via the user interface, etc., the preference of which can be set up in the user profile.

If the user does not enter the area of interest to be associated with the captured image, the image analysis engine can employ one or more image processing techniques to determine the area of interest of the user. In some embodiments, the image analysis engine may access information from a data store to assist in this determination. For example, the captured images may be compared to images with known static body (e.g., facial) features, such as the eyes, nose, and ears in order to determine the area of interest. In some embodiments, registration between captured images is performed to improve the analysis. This can be accomplished in some embodiments by referencing static body (e.g., facial) features present in each of the images to be analyzed. In some embodiments, one or more of these processes can be trained.

The example of the method 500 proceeds to block 506, where an image map is generated depicting changes to the area of interest over time. In some embodiments, image analysis engine determines or detects changes in one or more of size, shape, color, uniformity, etc., of existing lesions (e.g., moles, acne, dark sports, etc.), detects new lesions, detects the absence of previously detected lesions, detects a progression of a lesion, etc. In some embodiments, the image analysis engine compares and interprets the gross changes of the lesions over time so as to decide and flag (e.g., identify, highlight, mark, etc.) a subset of lesions as “suspicious.” The lesions that are flagged as suspicious have changed in size, shape, color, uniformity etc., an amount greater than a predetermined threshold (e.g., 1-3%, 2-4%, 3-5%, etc.). This subset of lesions can be represented in an image map in the form of a skin condition map or profile, etc. In some embodiments, the image analysis engine can identify the changes in the images as acne blemishes, or other skin conditions, which can also be represented in a skin condition map or profile, etc. The image map can be subsequently output via a display device.

Next, at block 508, a skin condition of the area of interest is determined based on the skin condition map or profile. In some embodiments, the skin condition engine 208 of the mobile computing device 104 or the skin condition engine 306 of the server computing device 108 analyzes the skin condition map or profile and determines, for example, the stages of acne for each region of the area of interest. In doing so, the skin condition engine can access data from the skin condition data store 218, 318. In some embodiments, the skin condition engine identifies a progression of a skin condition, such as acne (determined from an analyses of the images). In other embodiments, this step can be carried out, at least in part, by the image analysis engine. If the changes to certain areas (e.g., pixel groups) of the images match, for example, the progression of a known skin condition (e.g., an acne blemish) accessed from the skin condition data store, the skin condition engine (or optionally, the image analysis engine) can identify these groups of pixels as a blemish and can assigned the blemish a skin condition level (e.g., acne stage, etc.).

The example of the method 500 then proceeds to block 510, where a treatment protocol and/or product are recommended for each region of the area of interest based on the determined skin condition (e. g., stage of acne, etc.). In doing so, data can be accessed from the product data store 216, 316, user data store 214, 314, etc. Different products and/or treatment protocols can be recommended for regions with difference skin condition levels. Any recommendation generated by the recommendation engine can be presented to the user in any fashion via the user interface engine 210 on display 202. The recommendation can be saved in the user's profile in user data store 214, 314. In some embodiments, previous recommendations and/or treatments administered by the user can be used in the product and/or treatment protocol recommendation. In some embodiments, the efficacy of the recommendation can be tracked, which can be used to train the recommendation engine and/or data stored in the product data store for improved recommendations in subsequent uses.

The method 500 then proceeds to an end block and terminates.

The present application may reference quantities and numbers. Unless specifically stated, such quantities and numbers are not to be considered restrictive, but exemplary of the possible quantities or numbers associated with the present application. Further in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

The above description of illustrated examples of the present disclosure, including what is described in the Abstract, are not intended to be exhaustive or to be a limitation to the precise forms disclosed. While specific embodiments of, and examples for, the present disclosure are described herein for illustrative purposes, various equivalent modifications are possible without departing from the broader spirit and scope of the present disclosure, as claimed. Indeed, it is appreciated that the specific example voltages, currents, frequencies, power range values, times, etc., are provided for explanation purposes and that other values may also be employed in other embodiments and examples in accordance with the teachings of the present disclosure.

These modifications can be made to examples of the disclosed subject matter in light of the above detailed description. The terms used in the following claims should not be construed to limit the claimed subject matter to the specific embodiments disclosed in the specification and the claims. Rather, the scope is to be determined entirely by the following claims, which are to be construed in accordance with established doctrines of claim interpretation. The present specification and figures are accordingly to be regarded as illustrative rather than restrictive.

The embodiments of the disclosed subject matter in which an exclusive property or privilege is claimed are defined as follows:

1. A computer implemented method for determining changes in a skin condition of a subject, comprising:
obtaining a plurality of images of an area of interest associated with the subject, the plurality of images taken sequentially over time, wherein each image taken is separated in time by a time period;
determining one or more differences between the plurality of images.

2. The method of claim 1, further comprising
generating an image map of the area of interest, the image map indicative of the differences between the plurality of images.

3. The method of claim 2, further comprising
determining a skin condition based on the image map.

4. The method of claim 2, wherein the image map indicates changes in one or more of a size, a shape, a color, and uniformity of an object contained in the area of interest.

5. The method of claim 4, further comprising
recommending one of a treatment or a product based on the determined skin condition.

6. The method of claim 3, wherein the skin condition is selected from a group consisting of dermatitis, eczema, acne, and psoriasis.

7. The method of claim 1, wherein the time period is selected from the group consisting of 24 hours, one week, one month, two months, three months, four months, five months, six months, and one year.

8. The method of claim 1, further comprising
if the difference detected is greater than a preselected threshold value, notifying the user that a change has been detected.

9. The method of claim 1, further comprising
determining the area of interest based at least one the captured images.

10. A system for determining changes in a skin condition of a subject, comprising:
a camera configured to capture one or more images;
one or more processing engines including circuitry configured to:
cause the camera to capture one or more images of an area of interest associated with the subject, the one or more images taken sequentially over time so as to obtain a plurality of images separated in time by a time period selected from the group consisting of 24 hours, one week, one month, two months, three months, four months, five months, and six months, and one year;
determine one or more differences between the captured images, the differences indicative of changes in one or more of a size, a shape, a color, and uniformity of an object contained in the area of interest; and
determine a skin condition based on the determined differences or flagging the object for subsequent analysis if said differences are greater than a preselected threshold.

11. The system of claim 10, wherein the one or more processing engines include circuitry configured to:
determine the skin condition based on the determined differences; and
recommend a treatment protocol or a product based on the determined skin condition.

12. The system of claim 10, wherein the one or more processing engines includes circuitry configured to determine changes in one or more of: size, shape, color, uniformity of an existing lesion, detect new lesions, detect the absence of previously detected lesion(s), or detect a progression of a lesion.

13. The system of claim 10, wherein the one or more processing engines includes circuitry configured to:
detect a progression of a lesion from the detected differences in the plurality of images; and
determine one or more stages of the lesion based on the detected progression of the lesion.

14. The system of claim 10, wherein the one or more processing engines includes:
a user interface engine including circuitry configured to cause the camera to capture the plurality of images;
an image analysis engine including circuitry for comparing two or more images using a similar/difference algorithm to determine one or more differences between said images; and
a skin condition engine including circuity configured for analyzing an image map of the determined one or more differences to locate a lesion, and for determining the stage of the lesion located in the image map.

15. The system of claim 14, wherein the one or more processing engines further includes:
a recommendation engine including circuity configured to recommend a treatment protocol and/or product for each region based at least on the determined skin condition.

16. The system of claim 15, wherein the skin condition is selected from a group consisting of dermatitis, eczema, acne, and psoriasis.

17. A computer-implemented method for determining changes in a skin condition of a subject, the method comprising:
obtaining a plurality of images of an area of interest associated with the subject, the plurality of images taken sequentially over a time with each taken image separated in time by a time period;
determining a skin condition based on least the plurality of images;
determining at least one product recommendation based on at least the determined skin condition; and
providing the at least one product recommendation to the subject.

18. The computer-implemented method of claim 17, wherein said obtaining, by a first computing device, a plurality of images of an area of interest associated with the subject includes
capturing, by a camera of a first computing device, the plurality of images.

19. The computer-implemented method of claim 18, wherein said determining a skin condition based on least the plurality of images or said determining at least one product recommendation based on at least the determined skin condition is carried out by a second computing device remote from the first computing device.

20. The method of claim 19, wherein the skin condition is selected from a group consisting of dermatitis, eczema, acne, and psoriasis.
